(19) United States
(12) Patent Application Publication
(10) Pub. No.: US 2015/0100276 A1
(43) Pub. Date: Apr. 9, 2015

(54) MEDICATION RECORDING APPARATUS AND METHOD

(71) Applicant: National Applied Research Laboratories, Hsinchu City (TW)

(72) Inventors: Chun-Ming HUANG, Hsinchu City (TW); Chen-Chia CHEN, Hsinchu City (TW); Shu-Hui HUNG, Hsinchu City (TW)

(73) Assignee: National Applied Research Laboratories, Hsinchu City (TW)

(21) Appl. No.: 14/133,160

(22) Filed: Dec. 18, 2013

(30) Foreign Application Priority Data
Oct. 4, 2013 (TW) 102136039

(51) Int. Cl. A61M 15/00

(52) U.S. Cl. CPC A61M 15/008
USPC 702/187; 128/203.12; 128/203.15; 702/189

(57) ABSTRACT

A medication recording apparatus is used for motoring and recording patient's medication. The medication recording apparatus is coupled with a drug inhaler. When the patient receives a drug from the drug inhaler, a drug-supply actuation is generated. The medication recording apparatus has: a sensing module for sensing at least one type of the medication recording apparatus motion generated by the drug-supply actuation and sensing a motion data generated by the drug-supply actuation; a control and analysis module for determining if the medication recording apparatus motion is a valid medication according to the motion data and recording correct medication information corresponding to the correct medication. A medication recording method is also provided and used to analyze if the drug-supply actuation of the patient generates the valid medication and to record the correct medication information, so as to control the patient's medication status.

### CROSS-REFERENCE

This application claims priority of Taiwan application, Ser. NO. 102136039, filed Oct. 4, 2013, and entitled “Medication Recording Apparatus and Method”, by the same inventors.

### FIELD OF THE INVENTION

The present invention relates to a medication recording apparatus and method, and more particularly to a medication recording apparatus coupled with a drug inhaler to be taken by a patient for sensing if a drug-supply actuation is an correct medication or not and recording the correct medication for monitoring the patient's medication condition, and to a medication recording method by using the medication recording apparatus.

### BACKGROUND OF THE INVENTION

Most patients generally consider that their medication (medicine therapy) is in compliance with the instructions of physicians, but in fact, only a few patients can be surely medication according to the medication time, medication dose and correct medication indicated dosage. Therefore, that often tends to reduce medicine efficacy to the patients, even with the possibility of sudden death. Especially, it's necessary for chronic patients to be treated with long-term administration to control the condition. But, the chronic patients may not take the medicine within a prescribed time, or the mode of medication is not correct.

For example, many patients with chronic obstructive pulmonary disease (COPD) and asthma use a metered dose inhaler (MDI) or a dry powder inhaler (DPI), but they often ignore the importance of correct medication and thus maybe inhale multiple doses at one time, or inhale at the wrong time, or even have no medication for several days. Such wrong ways of medication will tend to reduce the medicine's therapeutic efficacy or no efficacy, or even have the potential risk of death.

Therefore, the following two traditional ways can help to detect if the patients use the inhalers in an correct medication manner or not: (1) observing and asking patients directly by the physicians, (2) using the number of used time of the inhaler and the remaining dosage of medicine to calculate if the patients use the inhalers in an correct medication manner or not. However, the second method only can calculate of the correct medication passively and can not confirm whether the patient take the medicine in accordance with the medication time, medication dose and correct medication indicated dosage. So far, for the patients using drugs inhalers, the physicians can not precisely track the patients' medication status correctly and accurately in clinical and can not further control the patients' medication status precisely. Therefore, there is a need to develop a medication recording apparatus and method to continuously monitor and determine whether the patients' medication is the correct medication or not in order to enhance the pharmaceutical efficacy of medicine and prevent from bursting into a severe illness.

### SUMMARY OF THE INVENTION

A primary object of the present invention is to provide a medication recording apparatus and method, wherein the medication recording apparatus is used for monitoring and recording a patient's medication behavior. The medication recording apparatus senses at least one type of the medication recording apparatus motion generated by the patient during medication. The medication recording apparatus helps to avoid incomplete medication, or insufficient medication dosage, and continuously monitors if the patient's medication behavior is correct or not, so as to maintain the patient in a correct medication during medical treatment.

To achieve the above object, the present invention provides a medication recording apparatus, for monitoring and recording a patient's medication condition and identifying if the medication behavior is correct or not, wherein the medication recording apparatus is coupled with a drug inhaler, when the patient receives a drug from the drug inhaler, at least one type of medication recording apparatus motion is generated by the patient's drug-supply actuation, the medication recording apparatus comprises: at least one sensing module installed in the drug inhaler for sensing the medication recording apparatus motion generated by the drug-supply actuation and sensing an motion data generated by the medication recording apparatus motion; a control and analysis module installed in the drug inhaler for analyzing and recording the motion data, wherein the control analysis module comprises a processing unit for determining if medication recording apparatus motion is the correct medication according to the motion data and for recording an correct medication information corresponding to the correct medication.

In the above-mentioned medication recording apparatus of the present invention, the sensing module is an accelerometer, a gyroscope, a magnetic sensor or any combination thereof.

In the above-mentioned medication recording apparatus of the present invention, the correct medication includes medication time and medication number.

In the above-mentioned medication recording apparatus of the present invention, the control and analysis module further comprises a storage unit, a communication unit and a system clock unit, the storage unit is used to store the correct medication information, the communication unit is used to transfer the correct medication information to an external device, and the system clock unit is used to provide time corresponding to the correct medication.

In the above-mentioned medication recording apparatus of the present invention, the medication recording apparatus further comprises a power supply unit for maintaining operations of the sensing module and the control and analysis module.

In the above-mentioned medication recording apparatus of the present invention, the drug inhaler is a metered dose inhaler, wherein the metered dose inhaler further comprises a drug container, the drug container has a supply end and a press end, and the medication recording apparatus is disposed on the drug container and the medication recording apparatus comprises a sleeved component for mounting onto the press end of the drug container.

In the above-mentioned medication recording apparatus of the present invention, the drug inhaler is a dry powder inhaler, wherein the dry powder inhaler comprises a movable component, the medication recording apparatus is coupled with the movable component for the patient to use the movable component to generate the medication recording apparatus motion.

To achieve the above object, the present invention further provides another medication recording apparatus, for sensing and recording a patient's medication behavior and identifying if the medication behavior is correct or not, wherein the medication recording apparatus is coupled with a drug inhaler, when the patient receives a drug from the drug inhaler, at least one type of medication recording apparatus is generated by the patient's drug-supply actuation, the medication recording apparatus comprises: at least one magnetic sensor installed in the drug inhaler for sensing changes of a magnetic field intensity; at least one magnetic component installed in the drug inhaler, and having a sensing distance apart from the magnetic sensor, wherein the drug-supply actuation actuates the magnetic component to change the sensing distance between the magnetic component and the magnetic sensor, so that the magnetic field intensity sensed by the magnetic sensor is synchronously changed to generate an motion data, or the magnetic component and the magnetic sensor is disposed on two opposite positions with the sensing distance therebetween and the drug-supply actuation changes the opposite positions between the magnetic component and the magnetic sensor to generate the motion data; and a control and analysis module installed in the drug inhaler for analyzing and recording the motion data, wherein the control analysis module comprises a processing unit for determining if the drug-supply actuation is the correct medication according to the motion data and for recording an correct medication information corresponding to the correct medication.

In the above-mentioned medication recording apparatus of the present invention, the correct medication includes correct medication time and correct medication number.

In the above-mentioned medication recording apparatus of the present invention, the medication recording apparatus further comprising a linkage component coupled with the magnetic component and actuated by the magnetic component, wherein the drug-supply actuation actuates the magnetic component and the linkage component to change the sensing distance.

In the above-mentioned medication recording apparatus of the present invention, the magnetic sensor is a Hall effect sensor, a Hall effect switch or any combination thereof.

In the above-mentioned medication recording apparatus of the present invention, the control and analysis module further comprises a storage unit, a communication unit and a system clock unit, the storage unit is used to store the correct medication information, the communication unit is used to transfer the correct medication information to an external device, and the system clock unit is used to provide time corresponding to the correct medication.

In the above-mentioned medication recording apparatus of the present invention, the medication recording apparatus further comprises a power supply unit for maintaining operations of the magnetic sensor and the control and analysis module.

In the above-mentioned medication recording apparatus of the present invention, the magnetic component and the magnetic sensor is disposed on two opposite positions with the sensing distance therebetween, and the drug-supply actuation changes the opposite positions between the magnetic component and the magnetic sensor.

In the above-mentioned medication recording apparatus of the present invention, the drug inhaler is a metered dose inhaler, wherein the metered dose inhaler further comprises a drug container, the drug container has a supply end and a press end, and the medication recording apparatus is disposed on the drug container, and the medication recording apparatus comprises a sleeved component for mounting onto the press end of the drug container.

In the above-mentioned medication recording apparatus of the present invention, the drug inhaler is a dry powder inhaler, wherein the dry powder inhaler comprises a movable component, the medication recording apparatus is coupled with the movable component for the patient to use the movable component to generate the medication recording apparatus motion.

To achieve the above object, the present invention provides a medication recording method, the method comprises steps of: using any one of the above-mentioned medication recording apparatus to analyze if a drug-supply actuation of a patient generates a correct medication or not; and recording the correct medication information corresponding to the correct medication.

To achieve the above object, the present invention further provides another medication recording method for sensing and recording at least one type of medication recording apparatus motion generated by a drug inhaler used by a patient, the method comprising steps of: (a) providing at least one sensing module installed in the drug inhaler for sensing the at least one type of medication recording apparatus motion generated by the patient's drug-supply actuation and sensing an motion data generated by medication recording apparatus motion; (b) providing a control and analysis module installed in the drug inhaler for analyzing the motion data and determining if the drug-supply actuation is the correct medication or not; and (c) if the drug-supply actuation is the correct medication, recording an effective medication information corresponding to the correct medication into a medication recording apparatus; and if the drug-supply actuation is not the correct medication, identifying the drug-supply actuation as an invalid medication and recording an invalid medication information corresponding to the invalid medication into the medication recording apparatus for reminding the patient.

The present invention provides a medication recording apparatus and method, for precisely controlling the patients' medication condition, and helping the patients to identify if the medication is an effective medication or not, so as to remind the patient to avoid improper medication.

### DESCRIPTION OF THE DRAWINGS

FIG. 1 is a perspective view of a medication recording apparatus according to a preferred embodiment of the present invention;

FIG. 2 is an exploded perspective view of the medication recording apparatus according to the preferred embodiment of the present invention;

FIG. 3 is a block diagram of the sensing module and the control and analysis module of the medication recording apparatus according to the preferred embodiment of the present invention;

FIG. 4 is a curve chart of the drug-supply result of using a metered dose inhaler of the medication recording apparatus according to the preferred embodiment of the present invention;

FIG. 5 is an exploded perspective view of a medication recording apparatus according to another preferred embodiment of the present invention;

FIG. 6 is a curve chart of the drug-supply result of using a dry powder inhaler of the medication recording apparatus according to another preferred embodiment of the present invention;

FIG. 7 is a perspective view of a medication recording apparatus according to further another preferred embodiment of the present invention;

FIG. 8 is an exploded perspective view of the medication recording apparatus according to further another preferred embodiment of the present invention;

FIG. 9 is a partially enlarged view of the location of a magnetic sensor and magnetic component of the medication recording apparatus according to further another preferred embodiment of the present invention;

FIG. 10 is a curve chart of the drug-supply result of using the metered dose inhaler of the medication recording apparatus according to further another preferred embodiment of the present invention;

FIG. 11 is a cross-sectional view of a medication recording apparatus according to still another preferred embodiment of the present invention, taken along a line A-A; and

FIG. 12 is a flowchart of a medication recording method according to the preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings.

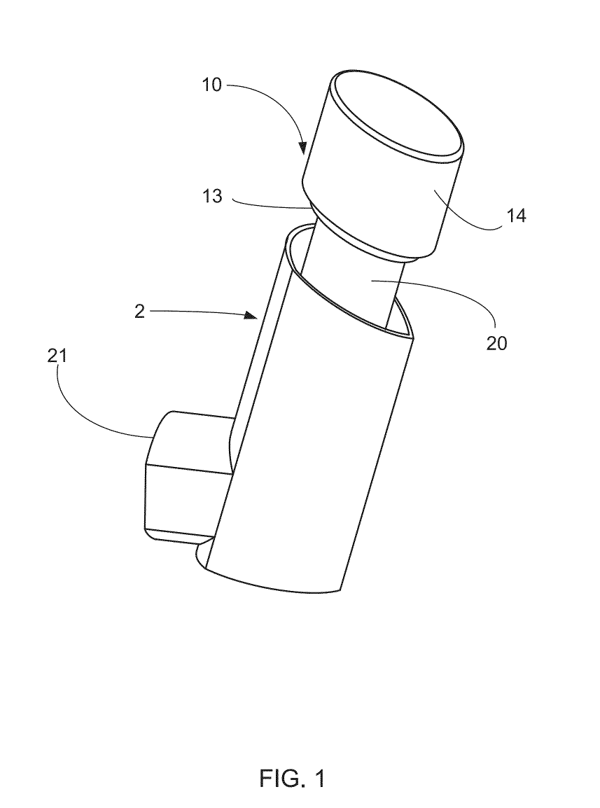

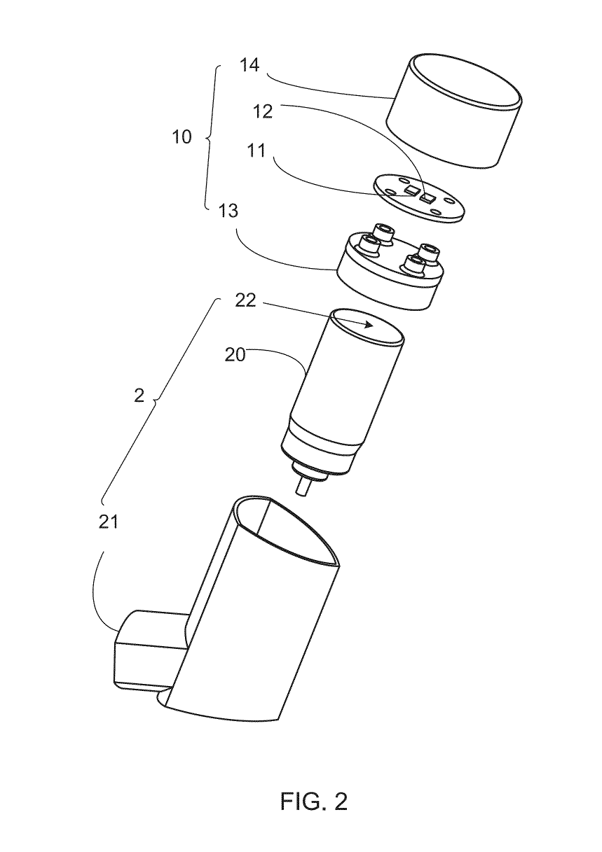

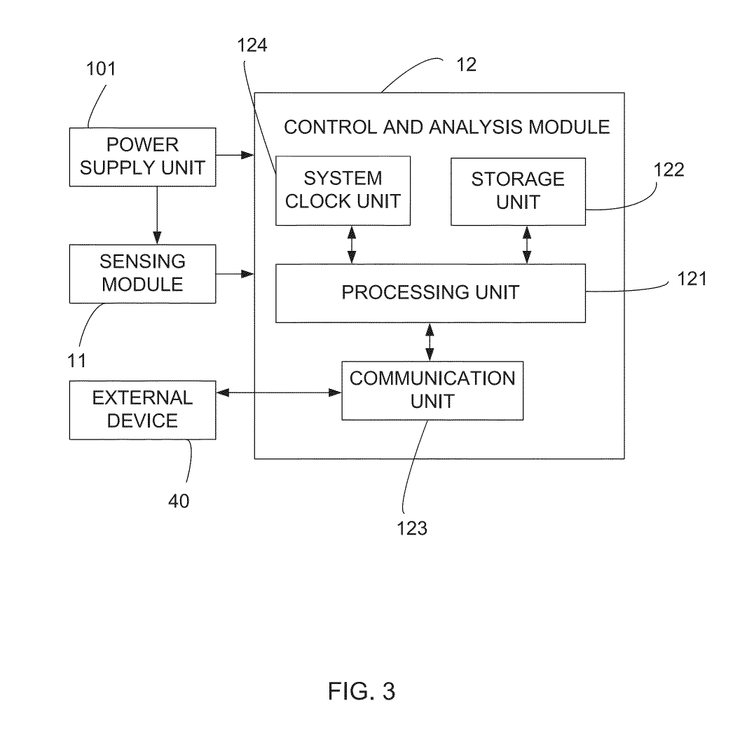

Referring now to FIG. 1 to FIG. 3, a perspective view and an exploded perspective view of a medication recording apparatus according to a first embodiment of the present invention are illustrated, wherein a medication recording apparatus 10 is used for motoring and recording a patient's medication behavior and identifying if the medication behavior is correct or not, and the medication recording apparatus 10 is coupled with a drug inhaler 2. When the patient receives a drug from the drug inhaler 2, at least one type of medication recording apparatus motion is generated by the patient's drug-supply actuation. The medication recording apparatus 10 comprises: at least one sensing module 11 and a control and analysis module 12, wherein the sensing module 11 is an accelerometer, a gyroscope, a magnetic sensor or any combination thereof, and used for sensing at least one type of medication recording apparatus generated by the drug inhaler 2. The sensing module 11 senses at least one type of medication recording apparatus motion generated by the patient using the drug inhaler 2 and is used for sensing a motion data corresponding to the medication recording apparatus motionact. The control and analysis module 12 analyzes and records the motion data, and comprises a processing unit 121 for determining if the drug-supply actuation is the correct medication according to the motion data and for recording the correct medication information corresponding to the correct medication, wherein the correct medication includes correct medication time and correct medication number.

In this embodiment of the present invention, the control and analysis module 12 further comprises a storage unit 122, a communication unit 123 and a system clock unit 124, wherein the storage unit 122 is used to store the correct medication information, the communication unit 123 is used to transfer the effective medication information to an external device 40, and the system clock unit 124 is used to provide time corresponding to the correct medication.

In this embodiment of the present invention, the medication recording apparatus 10 further comprises a power supply unit 101 for maintaining operations of the sensing module 11 and the control and analysis module 12.

In this embodiment of the present invention, the operation of the medication recording apparatus 10 is described, as follows: Firstly, mounting the medication recording apparatus 10 on the drug inhaler 2, wherein the setting method is to assemble the medication recording apparatus 10 inside the drug inhaler 2 or detachably mount the medication recording apparatus 10 on the outer surface of the drug inhaler 2, but not limited thereto. Secondly, when the patient uses the drug inhaler 2, the medication recording apparatus 10 is simultaneously turned on. The power supply unit 101 supplies the electricity for the medication recording apparatus 10. The sensing module 11 senses at least one type of medication recording apparatus motion generated by the patient using the drug inhaler 2, wherein the drug-supply actuation applied to the drug inhaler 2 can be press force or rotation force or any combination thereof. At this time, the sensing module 11 senses and records at least one type of medication recording apparatus motion form the patient using the drug inhaler 2, such as an actuation of pressing, rotating, squeezing, shaking, tilting or any combination thereof for generating the corresponding motion data.

Moreover, the processing unit 121 of the control and analysis module 12 analyzes the motion data immediately and determines if the drug-supply actuation is correct or not. If the drug-supply actuation is the correct medication, the control and analysis module 12 records the correct medication information corresponding to the correct medication into a medication recording apparatus 10, wherein the correct medication including effective medication time and correct medication number is stored in the storage unit 122. Besides, the correct medication information comprises the time corresponding to the correct medication and the motion data when the drug-supply actuation is determined as the correct medication. The effective medication information stored in the storage unit 122 can be transferred to the external device 40 by the communication unit 123. The external device 40 can be a smart phone, a tablet computer or related electronic products, and combined with the relevant application programs or software for reminding the patient the medication time and providing as a reference about medication condition for a physician. The process mentioned above is the operation of the medication recording apparatus 10 used by the patient.

As mentioned above, if the motion data of the medication recording apparatus motion exercised by the patient currently does not reach the threshold values of motion data corresponding to the correct medication, the drug-supply actuation exercised by the patient may be incomplete. The efficacy of this medication will be reduced or even lost due to the incomplete drug-supply actuation. Therefore, if the processing unit 121 of the control and analysis module 12 determines that the motion data of the medication recording apparatus motion exercised by the patient currently does not reach the threshold values of motion data corresponding to the correct medication, the drug-supply actuation is considered as an incorrect/invalid medication. The control and analysis module 12 identifies the drug-supply actuation as an invalid medication and records an invalid medication information corresponding to the invalid medication into the storage unit 122 of the medication recording apparatus 10 for reminding the patient, wherein the invalid medication information comprises invalid medication time and invalid medication number, and transfers to the external device 40 by the communication unit 123 to remind the patient about the incomplete medication.

In this embodiment of the present invention, the drug inhaler 2 is a metered dose inhaler. The metered dose inhaler comprises a drug container 20, the drug container 20 has a supply end 21 and a press end 22, and the medication recording apparatus 10 is disposed on the drug container 20. The medication recording apparatus 10 comprises a sleeved component 13 for mounting onto the press end 22 of the drug container 20. And, the medication recording apparatus 10 further comprises a cover 14 mounting onto the medication recording apparatus 10 for the drug-supply actuation forcing to the cover 14, so that the press end 22 is pressed to allow the metered dose inhaler to administrate the drug.

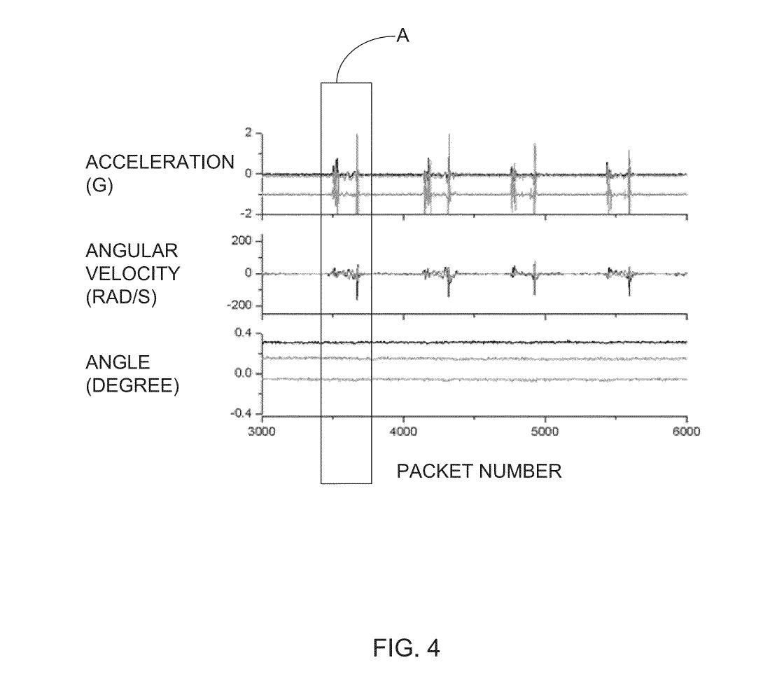

As mentioned above, the sensing module 11 can be used to record the medication recording apparatus motion generated by the patient using the drug inhaler 2, and then the control and analysis module 12 identifies the medication is correct or not. In this embodiment of the present invention, the drug inhaler 2 is preferably a metered dose inhaler of Berotec N 100 mcg/puff Metered Aerosol produced by Boehringer Ingelheim pharmaceutical company. When the patient uses the drug inhaler 2, the motion characteristics of the drug container 20 will be a linear reciprocating motion and vibration, and this is the medication recording apparatus motion generated by the metered dose inhaler from the patient's using. Referring to the FIG. 4, an area A in the figure is the time of the patient using Berotec N 100 mcg/puff Metered Aerosol, the sensing module 11 senses at least one type of medication recording apparatus motion.

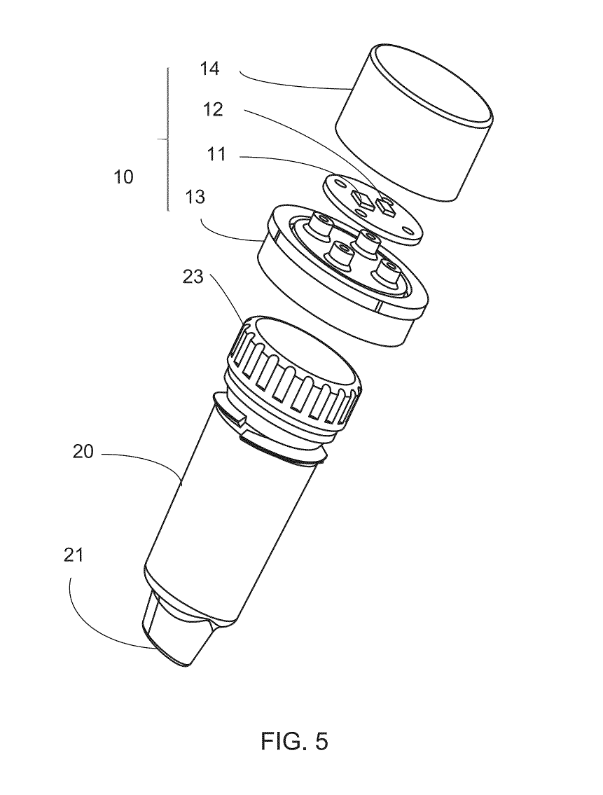

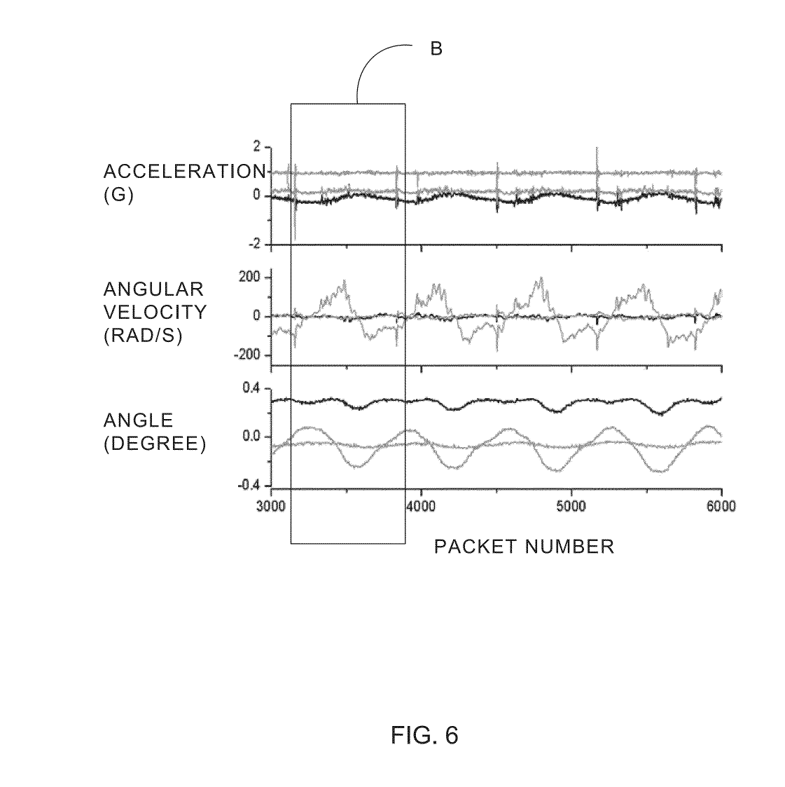

And referring to the FIG. 5, an exploded perspective view of a medication recording apparatus according to another preferred embodiment of the present invention is illustrated. In this embodiment of the present invention, the drug inhaler 2 is a dry powder inhaler, wherein the dry powder inhaler comprises a switch component 23, and the medication recording apparatus 10 is coupled with the movable component 23 for the patient to use the movable component 23 to generate the medication recording apparatus motion. And, the medication recording apparatus 10 further comprises a cover 14 mounting onto the medication recording apparatus 10. In this embodiment of the present invention, the drug inhaler 2 is preferably a dry powder inhaler of Symbicort® Turbuhaler produced by Astrazeneca pharmaceutical company. When the patient uses the drug inhaler 2, the motion characteristics of the movable component 23 of the drug container 20 generates a rotary motion centered on the axial center of the switch component 23 and generates a vibration, and this is the drug-supply actuation generated by the dry powder inhaler. Referring to the FIG. 6, an area B in the figure is the time of the patient using Symbicort® Turbuhaler, the sensing module 11 senses at least one type of medication recording apparatus motion.

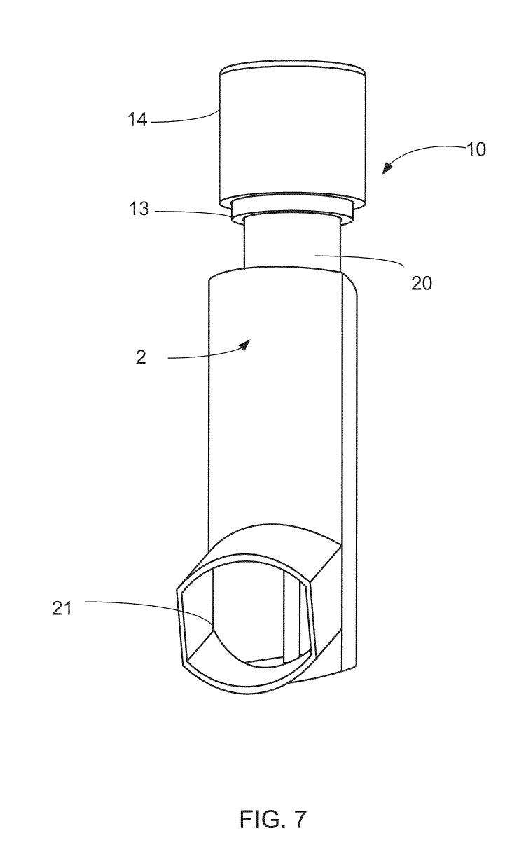

Referring to FIG. 7 to FIG. 9, a medication recording apparatus 10 according to further another preferred embodiment of present invention is illustrated. The medication recording apparatus 10 is used for sensing and recording a patient's medication behavior and identifying if the medication behavior is correct or not. The medication recording apparatus 10 is coupled with a drug inhaler 2. When the patient receives a drug from the drug inhaler 2, at least one type of medication recording apparatus motion is generated by the patient's drug-supply actuation. The medication recording apparatus 10 comprises at least one magnetic sensor 31, at least one magnetic component 32 and the control and analysis module 12. The magnetic sensor 31 installed in the drug inhaler 2 is used for sensing changes of a magnetic field intensity. The control and analysis module 12 is installed in the drug inhaler 2 nearby the magnetic sensor 31. The magnetic component 32 has a sensing distance 301 apart from the magnetic sensor 31. The drug-supply actuation actuates the magnetic component 32 to change the sensing distance between the magnetic component 32 and the magnetic sensor 31, so that the magnetic field intensity sensed by the magnetic sensor 31 is synchronously changed to generate the motion data. The control and analysis module 12 analyzes and records the motion data, wherein the control analysis module 12 comprises the processing unit 121 for determining if the drug-supply actuation is the correct medication according to the motion data and for recording the correct medication information corresponding to the correct medication.

In this embodiment of the present invention, the control and analysis module 12 further comprises a storage unit 122, a communication unit 123 and a system clock unit 124, and the structure and function of the components is the same with the above-described embodiment and will not be repeated here.

Similarly, as described in the foregoing embodiment, the medication recording apparatus 10 further comprises a power supply unit 101 for maintaining operations of the magnetic sensor 31 and the control and analysis module 12.

In this embodiment of the present invention, the medication recording apparatus 10 further comprising a linkage component 33 coupled with the magnetic component 32 and actuated by the magnetic component 32, wherein the drug-supply actuation actuates the magnetic component 32 and the linkage component 33 to change the sensing distance 301, so that the magnetic field intensity sensed by the magnetic sensor 31 is synchronously changed. Besides, the magnetic sensor 31 is a Hall effect sensor, a Hall effect switch or any combination thereof.

Similarly, as described in the foregoing embodiment, the drug inhaler 2 is a metered dose inhaler. The metered dose inhaler comprises the drug container 20, the drug container 20 has the supply end 21 and the press end 22, and the medication recording apparatus 10 is disposed on the press end 22 of the drug container 20. The medication recording apparatus 10 comprises a sleeved component 13 for mounting onto the press end 22 of the drug container 20. And, the medication recording apparatus 10 further comprises a cover 14 mounting onto the medication recording apparatus 10 for the drug-supply actuation forcing to the cover 14, so that the press end 22 is pressed to allow the metered dose inhaler to administrate the drug.

As mentioned above, when the drug inhaler 2 is not in use, the magnetic component 32 has the sensing distance 301 apart from the magnetic sensor 31. When the drug inhaler 2 is used by the patient, the patient presses the cover 14 and the press end 22 of the drug container 20 is pressed indirectly so as to release the medicine of the drug container 20. The linkage component 33 is compressed to actuate the magnetic component 32. At this time, the sensing distance 301 between the magnetic component 32 and the magnetic sensor 31 is shortened. After releasing the medicine, the patient stops to press the cover 14, the linkage component 33 returns an initial state, and the sensing distance 301 between the magnetic component 32 and the magnetic sensor 31 is also returned to its initial state.

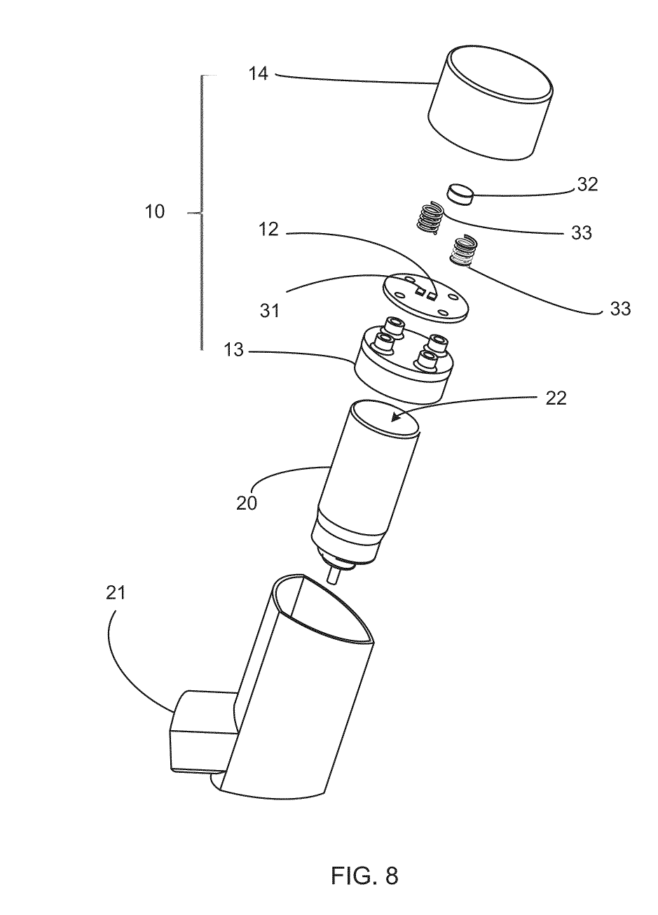

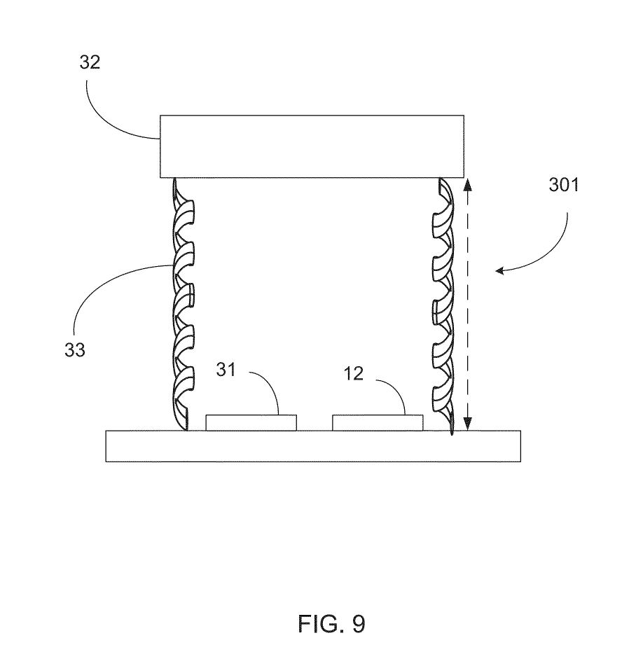

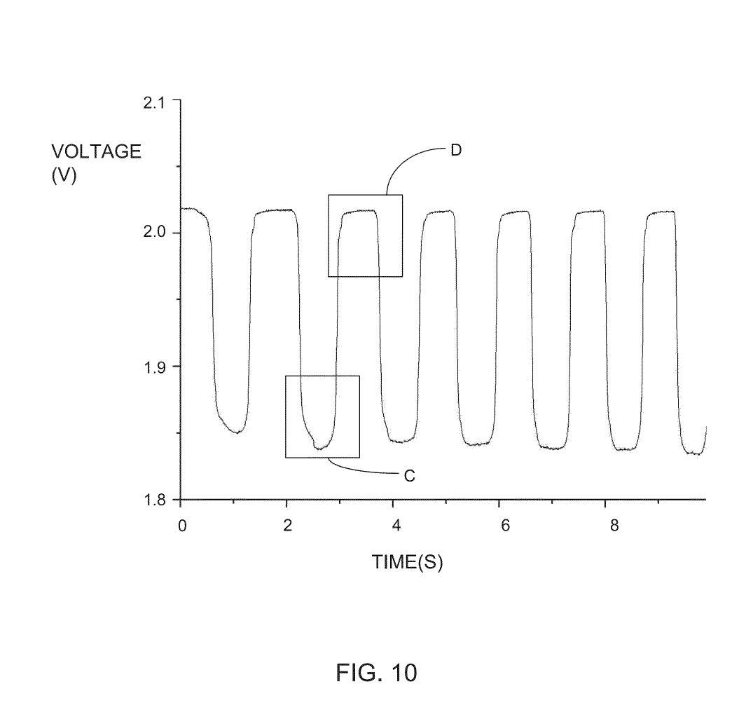

Referring to FIG. 10 and still referring to FIG. 8 and FIG. 9, the medication recording apparatus 10 is disposed on the press end 22 of the drug container 20. The patient presses the cover 14 as the drug-supply actuation, so as to force the press end 22 of the drug container 20 and thus generate at least one type of medication recording apparatus motion. The drug-supply actuation is to press the cover 14 of the medication recording apparatus 10, wherein the magnetic component 32 is pressed together with the cover 14. The magnetic component 32 is compressed so as to shorten the sensing distance 301 between the magnetic component 32 and the magnetic sensor 31. Thus, the magnetic sensor 31 senses the increasing of the magnetic field intensity as the medication recording apparatus motion, and correspondingly generates the motion data. The motion data is analyzed by the processing unit 121 of the control and analysis module 12. As shown in FIG. 10, an area C in the figure is shown that the press end 22 is pressed and the motion data is generated, an area D in the figure is shown that the press end 22 is released and generates a returning force. When the magnetic field intensity reaches to a threshold value of magnetic field intensity corresponding to achieve the correct medication, the processing unit 121 records the correct medication information corresponding to the correct medication into the storage unit 122, wherein the correct medication includes correct medication time and correct medication number. The following operation is the same as the foregoing embodiment and will not be repeated here.

As mentioned above, when the drug-supply actuation is finished at that time, the patient stops to press the cover 14, and the magnetic component 32 is not compressed, so as to allow the sensing distance 301 between the magnetic component 32 and the magnetic sensor 31 to return the initial distance. The magnetic sensor 31 senses the decreasing of the magnetic field intensity, and the control and analysis module 12 stops to analyze the drug-supply actuation as the correct medication. The following operation is the same as the foregoing embodiment and will not be repeated here.

Therefore, in this embodiment of the present invention, the linkage component 31 is located between the magnetic sensor 31 and the magnetic component 32. The linkage component 31 can be a spring, but the present invention is not limited thereto. The spring is coupled with the magnetic component 32 and actuated by the magnetic component 32. The spring and the magnetic component 32 is compressed by the patient pressing the cover 14 of the medication recording apparatus 10; or the spring and the magnetic component 32 is released by the patient stopping to press the cover 14 of the medication recording apparatus 10. Thus, the sensing distance 301 between the magnetic component 32 and the magnetic sensor 31 can be shortened or returned to the initial distance, and the magnetic sensor 31 further senses the increasing or decreasing of the magnetic field intensity for the control and analysis module 12 to determine if the drug-supply actuation is the correct medication or not.

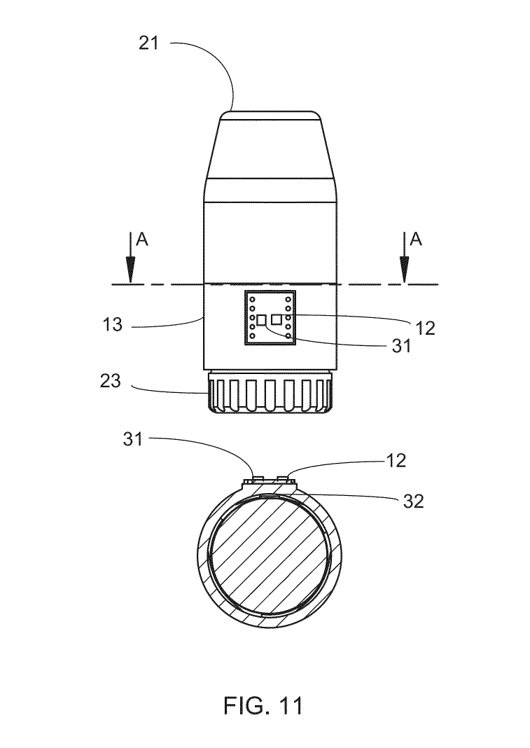

Moreover, referring to FIG. 11, a side view and a cross-sectional view of the medication recording apparatus according to still another preferred embodiment of the present invention, taken along a line A-A, are illustrated. The drug inhaler is a dry powder inhaler. The dry powder inhaler comprises the switch component 23. The patient rotates the movable component 23 to correspondingly generate the drug-supply actuation so as to supply the medicine. The medication recording apparatus 10 comprises a sleeved component 13 for mounting onto the movable component 23. At least one magnetic sensor 31 and the control and analysis module 12 are installed on a sidewall of the sleeved component 13. Besides, at least one magnetic component 32 is installed on a sidewall of the drug container 20. The magnetic component 32 and the magnetic sensor 31 are disposed on two opposite positions and can change the positions with each other. The patient rotates the movable component 23 to correspondingly generate the drug-supply actuation so as to drive the magnetic sensor 31. That motion changes the opposite positions between the magnetic component 32 and the magnetic sensor 31. The magnetic sensor 31 senses the changes of the magnetic field intensity. The following operation is the same as the foregoing embodiment and will not be repeated here.

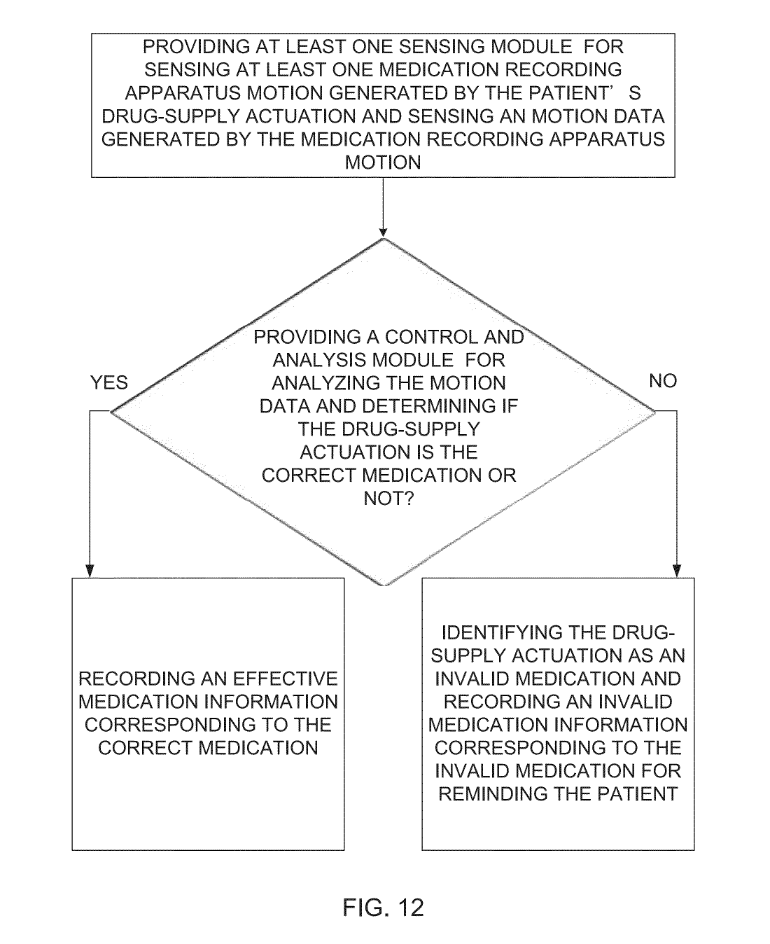

Referring to FIG. 12, a flowchart of a medication recording method according to the preferred embodiment of the present invention is illustrated. The method is used for sensing and recording at least one type of medication recording apparatus motion generated by a drug inhaler used by a patient, and the method comprises steps of: (a) providing at least one sensing module installed in the drug inhaler for sensing the at least type of medication recording apparatus motion generated by the patient's drug-supply actuation and sensing an motion data generated by the medication recording apparatus motion; (b) providing a control and analysis module installed in the drug inhaler for analyzing the motion data and determining if the drug-supply actuation is the correct medication or not; and (c) if the drug-supply actuation is the correct medication, recording an correct medication information corresponding to the correct medication into a medication recording apparatus; and if the drug-supply actuation is not the correct medication, identifying the drug-supply actuation as an invalid medication and recording an invalid medication information corresponding to the invalid medication into the medication recording apparatus for reminding the patient.

In this embodiment of the present invention, the sensing module is an accelerometer, a gyroscope, a magnetic sensor or any combination thereof, and used for sensing and recording at least one motion type or force type of drug-supply actuation.

In this embodiment of the present invention, if the control and analysis module determines the drug-supply actuation as the correct medication, the control and analysis module records the correct medication information corresponding to the correct medication in the storage unit. The correct medication information comprises the time corresponding to the correct medication and the system clock and to the motion data and type of drug-supply actuation when the drug-supply actuation is determined as the correct medication. The correct medication information stored in storage unit can transfer to the external device by the communication unit. The external device 40 can be a smart phone, tablet computer or related electronic products, and combines with the relevant application programs or software for reminding the patient the medication time and providing as a reference about medication condition for a physician.

In this embodiment of the present invention, if the control and analysis module determines the drug-supply actuation as the invalid medication, the storage unit still stores the invalid medication information corresponding to the invalid medication into the storage unit. The invalid medication information stored in the storage unit can be transferred to the external device by the communication unit for reminding the patient.

As described above, the technical features of the present invention is to provide the at least one sensing module for sensing at least one medication recording apparatus motion generated by the patient's drug-supply actuation, and provide the control and analysis module for determining if the drug-supply actuation is the correct medication or not. The technical features of the present invention can prevent the patient from the incomplete, or insufficient medication dosage, or without following the correct medication indicated dosage, so as to remind the patient to take the medicine and/or provide a reference data about medication condition for a physician to continuously monitor the patient's medication condition when the patient returns to the clinic.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A medication recording apparatus, for monitoring and recording a patient's medication behavior and identifying if the medication behavior is correct or not, wherein the medication recording apparatus is coupled with a drug inhaler, when the patient receives a drug from the drug inhaler, at least one type of medication recording apparatus motion is generated by a patient's drug-supply actuation, the medication recording apparatus comprising:
at least one sensing module installed in the drug inhaler for sensing the medication recording apparatus motion generated by the drug-supply actuation; and sensing an motion data generated by the medication recording apparatus motion;
a control and analysis module installed in the drug inhaler for analyzing and recording the motion data, wherein the control analysis module comprises a processing unit for determining if the drug-supply actuation is the correct medication according to the motion data and for recording an correct medication information corresponding to the correct medication.

2. The medication recording apparatus according to claim 1, wherein the sensing module is an accelerometer, a gyroscope, a magnetic sensor or any combination thereof.

3. The medication recording apparatus according to claim 1, wherein the correct medication includes correct medication time and correct medication number.

4. The medication recording apparatus according to claim 1, wherein the control and analysis module further comprises a storage unit, a communication unit and a system clock unit, the storage unit is used to store the correct medication information, the communication unit is used to transfer the correct medication information to an external device, and the system clock unit is used to provide time corresponding to the correct medication.

5. The medication recording apparatus according to claim 1, wherein the medication recording apparatus further comprises a power supply unit for maintaining operations of the sensing module and the control and analysis module.

6. The medication recording apparatus according to claim 1, wherein the drug inhaler is a metered dose inhaler, the metered dose inhaler further comprises a drug container, the drug container has a supply end and a press end, and the medication recording apparatus is disposed on the drug container.

7. The medication recording apparatus according to claim 6, wherein the medication recording apparatus comprises a sleeved component for mounting onto the press end of the drug container.

8. The medication recording apparatus according to claim 1, wherein the drug inhaler is a dry powder inhaler, the dry powder inhaler comprises a movable component, and the medication recording apparatus is coupled with the movable component for the patient to use the movable component to generate the medication recording apparatus motion.

9. A medication recording apparatus, for sensing and recording a patient's medication behavior and identifying if the medication behavior is correct or not, wherein the medication recording apparatus is coupled with a drug inhaler, when the patient receives a drug from the drug inhaler, at least one type of medication recording apparatus motion is generated by the patient's drug-supply actuation, the medication recording apparatus comprising:
at least one magnetic sensor installed in the drug inhaler for sensing changes of a magnetic field intensity;
at least one magnetic component installed in the drug inhaler, and having a sensing distance apart from the magnetic sensor, wherein the drug-supply actuation actuates the magnetic component to change the sensing distance between the magnetic component and the magnetic sensor, so that the magnetic field intensity sensed by the magnetic sensor is synchronously changed to generate an motion data; and
a control and analysis module installed in the drug inhaler for analyzing and recording the motion data, wherein the control analysis module comprises a processing unit for determining if the drug-supply actuation is the correct medication according to the motion data and for recording an correct medication information corresponding to the correct medication.

10. The medication recording apparatus according to claim 9, wherein the correct medication includes correct medication time and correct medication number.

11. The medication recording apparatus according to claim 9, wherein the control and analysis module further comprises a storage unit, a communication unit and a system clock unit, the storage unit is used to store the correct medication information, the communication unit is used to transfer the correct medication information to an external device, and the system clock unit is used to provide time corresponding to the correct medication.

12. The medication recording apparatus according to claim 9 wherein the medication recording apparatus further comprises a power supply unit for maintaining operations of the magnetic sensor and the control and analysis module.

13. The medication recording apparatus according to claim 9, further comprising a linkage component coupled with the magnetic component and actuated by the magnetic component, wherein the drug-supply actuation actuates the magnetic component and the linkage component to change the sensing distance.

14. The medication recording apparatus according to claim 9, wherein the magnetic component and the magnetic sensor is disposed on two opposite positions with the sensing distance therebetween, and the drug-supply actuation changes the opposite positions between the magnetic component and the magnetic sensor.

15. The medication recording apparatus according to claim 9, wherein the magnetic sensor is a Hall effect sensor, a Hall effect switch or any combination thereof.

16. The medication recording apparatus according to claim 9, wherein the drug inhaler is a metered dose inhaler, the metered dose inhaler further comprises a drug container, the drug container has a supply end and a press end, and the medication recording apparatus is disposed on the drug container.

17. The medication recording apparatus according to claim 16, wherein the medication recording apparatus comprises a sleeved component for mounting onto the press end of the drug container.

18. The medication recording apparatus according to claim 11, wherein the wherein the drug inhaler is a dry powder inhaler, the dry powder inhaler comprises a movable component, the medication recording apparatus is coupled with the movable component for the patient to use the movable component to generate the medication recording apparatus motion.
